(19) United States
(12) Patent Application Publication
(10) Pub. No.: US 2024/0148534 A1
(43) Pub. Date: May 9, 2024

(54) AIRBAG TYPE ELECTRIC TOE CORRECTION DEVICE

(71) Applicant: Hangzhou Moenmedic Health Technology Co., Ltd, Hangzhou City (CN)

(72) Inventor: Junfei Jiang, Hangzhou City (CN)

(21) Appl. No.: 18/416,601

(22) Filed: Jan. 18, 2024

Related U.S. Application Data
(63) Continuation-in-part of application No. 17/534,469, filed on Nov. 24, 2021.

(30) Foreign Application Priority Data
Mar. 3, 2023 (CN) 202320379521.7

(51) Int. Cl. A61F 5/01

(52) U.S. Cl. CPC A61F 5/019; A61F 5/012

(57) ABSTRACT

The invention belongs to the technical field of health care products, especially refers to an airbag type electric toe correction device, comprising the main support with hollow interior, the main support and auxiliary support being detachably connected; below the joint of main support and auxiliary support are equipped with an airbag, which is connected with pneumatic pump inside main support; the airbag is a multi-layer structure, when inflated, adjacent two layers of airbag form a V-shape gap; included angle formed by fixing portion and correction portion is designed reasonably and auxiliary support with different angle can be changed according to user's use time, which can meet different users' need and won't cause overcorrection or poor correction effect of user's toe; the airbag through the design of multi-layer structure can be made to expand with longer length and more compact, to ensure that the airbag more fit user's foot.

### 1. TECHNICAL FIELD

The invention belongs to the technical field of toe correction, especially refers to an airbag type electric toe correction device

### 2. BACKGROUND ART

At present, toe deformities are caused by people non-standard wearing shoes or ill-fitting shoes or other reasons, especially women wearing high-heeled shoes for a long period of time, causing hallux varus, therefore toe corrector is needed for correcting the symptoms of hallux varus by turning out the big tole.

For example, a toe corrector convenient to disassemble in Publication No. CN214342832U, which discloses that “The invention belongs to the technical field of medical auxiliary instruments, and particularly relates to a toe corrector convenient to disassemble, which comprises a main support, and a main bandage is arranged on the main support and is used for binding the main support on a foot arch; the auxiliary support is rotationally arranged on the main support; an auxiliary bandage is arranged on the auxiliary support and is used for binding the auxiliary support on the toe; a rotating column is arranged on the main support, and an elastic clamp is arranged on the auxiliary support. The main support and the auxiliary support are connected by enabling the elastic clamp to be clamped on the rotating column. The auxiliary support can be easily detached from the main support through the clamping rotation mode of the auxiliary support and the main support, so that the auxiliary support is easy to replace, and the toes with different deformity degrees can be adapted by replacing the auxiliary supports with different offset angles when the toes are used.

As can be seen from the above patent, what the existing toe corrector adopts is to adjust the auxiliary support on the toe corrector to meet different correction needs of the user by manually replacing the auxiliary support at different angles, however, the angle design of the auxiliary support of some toe corrector is not reasonable enough and will cause excessive correction of the user's toe by over-expansion, which may cause the user's injury, or due to the irrational design of the angle, the toe correction effect of the user is poor; furthermore, the airbag design is not reasonable enough, and when the airbag is inflated, the angle cannot be flexibly adjusted, thereby the comfort of the user can not be guaranteed; and lastly, it doesn't equipped with a display of inflated state of the airbag, the user is not able to accurately understand the inflation extent of the airbag, thereby not being conducive to effective toe correction and treatment.

### 3. SUMMARY OF THE INVENTION

The invention aims to provide an airbag type electric toe correction device.

The purpose of the invention is realized through the following:

An airbag type electric toe correction device, comprising the main support with hollow interior, the main support and auxiliary support being detachably connected; below the joint of main support and auxiliary support are equipped with an airbag, which is connected with the pneumatic pump inside the main support; the airbag is a multi-layer structure, when the airbag is inflated, adjacent two layers of the airbag form V-shape gaps;

As a further improvement, said auxiliary support comprises a fixing portion and a correction portion, one end of said fixing portion is provided with a circular opening to be clamped with the column-shaped body provided on the main support.

As a further improvement, the left and right sides of the opening in said auxiliary support are hollowed out to form hollow slots, and said auxiliary support is also provided with a through strap fixing groove in long strip shape, and said strap fixing groove is tied with a big toe strap.

As a further improvement, the included angle formed by said fixing portion and correction portion is from 0 to 15 degrees.

As a further improvement, the included angle formed by said fixing portion and correction portion is from 16 to 30 degrees.

As a further improvement, said auxiliary support is provided with a circular display.

As a further improvement, the air outlet of said pneumatic pump is connected to the airbag via air tube; said pneumatic pump is connected to the circuit board; below the circuit board is connected with the battery, and a control button is provided above said circuit board.

As a further improvement, a charging port is provided at the rear of said circuit board, and a solenoid valve is provided on one side of said pneumatic pump.

As a further improvement, on the left and right sides of said main support are symmetrically provided with strap fixing rings, on which are tied with foot strap.

As a further improvement, one side of said main support is sunken inwardly so as to fit the side of the foot of human body, one side of said auxiliary support is sunken inwardly so as to fit the big toe, and one side of said main support is connected to a flexible and soft spacer.

As a further improvement, the side of said spacer that fits the foot of the human body is provided with a vibration motor, which is a micro vibration motor and located inside the spacer so that the spacer protrudes in the direction of foot of the human body so as to better contact the foot of the human body.

Compared with the prior art, the invention has following advantages:

(1) The included angle formed by the fixing portion and correction portion is designed reasonably and auxiliary support with different angle can be changed according to user's use time, which can meet different users' need and won't cause overcorrection or poor correction effect of user's toe;

(2) The airbag is a multi-layer structure, when the airbag is inflated, adjacent two layers of the airbag form V-shape gaps, the airbag through the design of multi-layer structure can be made to expand with longer length and more compact, the gap of which can ensure that the airbag can expend to one side in the using process when the airbag is inflated so as to make sure that the airbag more fit user's foot, thereby enhancing comfort of user's orthodontic process and experience feeling.

(3) The auxiliary support is provided with a circular display, and the digital settings shown on the display allow the user to clearly know the expansion degree of the airbag, thus facilitating the user to adjust to the setting that is comfortable and has the best corrective effect.

(4) The side of said spacer that fits the foot of the human body is provided with a vibration motor, which is a micro vibration motor and located inside the spacer so that the spacer protrudes in the direction of foot of the human body so as to better contact the foot of the human body; and since the micro vibration motor can better contact the foot, it can improve the vibration effect so as to allow the user's foot to be more comfortable and relaxed.

### 4. BRIEF DESCRIPTION OF ACCOMPANY DRAWINGS

FIG. 1 is the exploded view of invention.

FIG. 2 is the interior structural diagram of the main support of the invention.

FIG. 3 is the three-dimensional structural diagram of the invention.

FIG. 4 is the partial enlarged view of the invention.

FIG. 5 is the diagram of the remote control of the invention.

### 5. SPECIFIC EMBODIMENT OF THE INVENTION

The invention is further described in detail hereinafter with reference to the specific embodiment.

As shown in Embodiment 1 and 3, an airbag type electric toe correction device, comprising the main support 1 with hollow interior, the main support 1 and auxiliary support 2 being detachably connected; below the joint of main support 1 and auxiliary support 2 are equipped with an airbag 3, through the inflation and expansion of the airbag 3, the auxiliary support 2 is forced to rotate to the outside, so as to be able to rotate the big toe tied to the auxiliary support 2 to the outside of the foot, thus realizing corrective effect of turning outward the big toe with varus deformity; The above utilizes expansion degree of airbag 3 to realize the effect of different degrees of toe's outward turning; since the expansion degree of the airbag 3 is easy to regulate and the size of the airbag 2 is easy to control arbitrarily, more fine rotation angle of the toe can also be realize realized, so as to facilitate the user to find the most suitable correction setting as well as the best correction effect; the airbag 3 is inflated by the pneumatic pump 4 inside the main support 1.

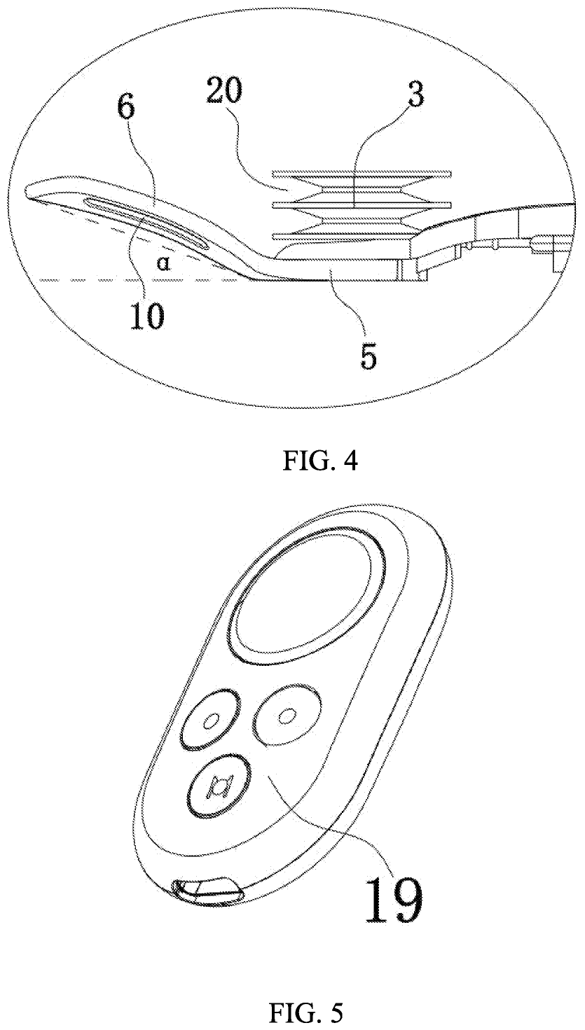

As shown in FIG. 4, the airbag 3 is a multi-layer structure, when the airbag 3 is inflated, adjacent two layers of the airbag 3 form V-shape gaps 20; the gaps 20 enable the airbag 3, which is in the form of column when inflated, to be bent in any direction, thereby ensuring that the thrust generated by the airbag 3 during its inflating process is completely exerted on one side of the user's foot, and that the bottom of airbag 3 is easy to firmly fit with the user's foot closely, thereby avoiding discomfort of the user's foot due to ill-fitting of the airbag 3 when it is inflated, and thus making the user's foot to be more comfortable when using the product, enhancing the user experience.

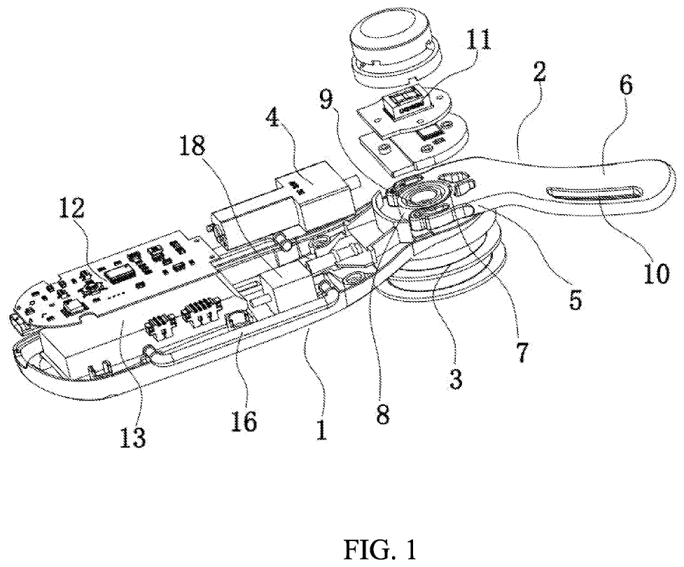

As shown in FIG. 1, said auxiliary support 2 comprises a fixing portion 5 and a correction portion 6, one end of said fixing portion 5 is provided with a circular opening 7 to be clamped with the column-shaped body 8 provided on the main support 1; Adopting such detachable clamping method can facilitate the user to change auxiliary support 2 with different specifications arbitrarily; the left and right sides of the opening 7 in said auxiliary support 2 are hollowed out to form hollow slots 9, which can enhance the elasticity of the left and right sides of the opening 7 and further facilitate the user to clamp the auxiliary support 2 to the main support 1 or detach auxiliary support 2 from the main support 1 more smoothly; said auxiliary support 2 is also provided with a through strap fixing groove 10 in long strip shape, and said strap fixing groove 10 is tied with a big toe strap 21 for fixing the user's big toe; while on the left and right sides of said main support 1 are symmetrically provided with strap fixing rings 16, on which are tied with foot strap 22 to be fixed on the foot of the user.

As shown in FIG. 1, said auxiliary support 2 is provided with a circular display 11, and the digital settings shown on the display 11 allow the user to clearly know the expansion degree of the airbag 3, thus facilitating the user to adjust to the setting that is comfortable and has the best corrective effect; the air outlet of the pneumatic pump 4 inflates the airbag 3 through air tube, the pneumatic pump 4 is connected to the circuit board 12, the battery 13 below the circuit board 12 supplies power to the entire device, a control button 14 is provided above said circuit board 12, which allows the user to control the expansion degree of the airbag 3 by pressing the control button 14.

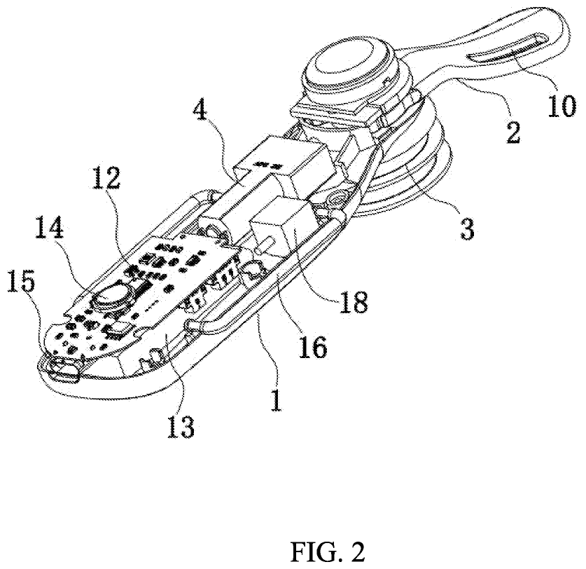

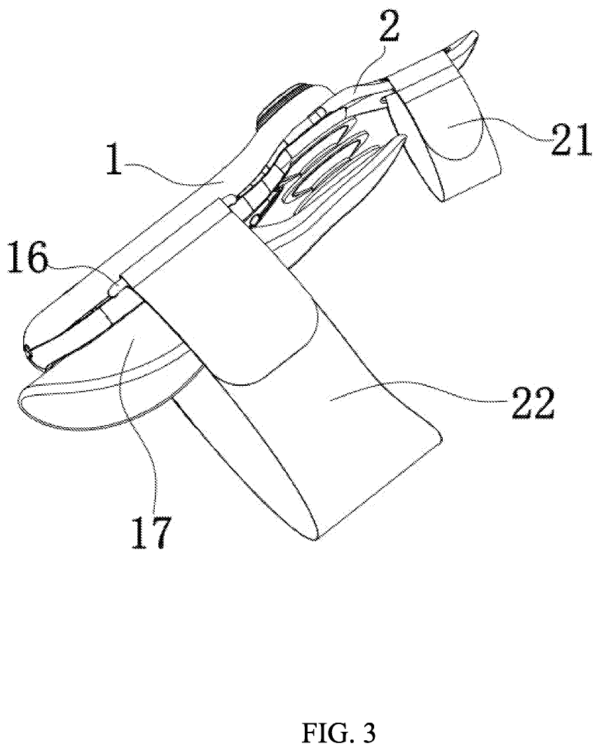

As shown in FIGS. 1, 2 and 5, the user uses the charging port 15 provided at the rear of said circuit board 12 for charging the internal battery 13; in addition, said circuit board 12 fits into the remote control, the user can also control the airbag through said remote control; a solenoid valve 18 is provided on one side of said pneumatic pump 4; one side of said main support 1 is sunken inwardly so as to fit the side of the foot of human body, the inwardly sunken structure allows the main support 1 to fit more closely to the side of the user's foot; one side of said auxiliary support 2 is sunken inwardly so as to fit the big toe, thereby making the user feel more comfortable in the using process; at the same time, one side of said main support 1 is connected to a flexible and soft spacer 17, which can enhance the comfort degree after the user wearing the device.

The side of said spacer 17 that fits the foot of the human body is provided with a vibration motor, which is a micro vibration motor and located inside the spacer 17 so that the spacer 17 protrudes in the direction of foot of the human body so as to better contact the foot of the human body; since the micro vibration motor can better contact foot, the vibration effect thereof is better and make the user feel more comfortable and relaxed.

As shown in FIG. 4, the included angle formed by said fixing portion and correction portion is from 0 to 15 degrees. As a preferred option, when the user begins to use the device, the user can first use the auxiliary support 2 with a large angle of 15 degrees, and after a long period of correction, the user can choose the auxiliary support 2 with a smaller angle, such as the auxiliary support 2 with 7.5 degree angle, and after correction with the auxiliary support 2 with 7.5 degree angle, the user can finally use the auxiliary support 2 with 0 degree angle for correction; the user's toes can be treated gradually with auxiliary supports 2 of different angles mentioned above, which makes the user not only get good correction of the toe, but also not feel uncomfortable during the correction process; in addition, the user with serious toe deformation can also be corrected with a of 16 to 30 degrees first, so as not to cause damage or pain to the user's toe.

The invention and its embodiments have been described above, but the description is not limited thereto; only one embodiment of the invention is shown in the drawings, and the actual structure is not limited thereto. In general, it is to be understood by those skilled in the art that non-creative design of structural forms and embodiments that are similar to the technical solutions without departing from the spirit of the invention shall all fall within the protective scope of the invention.

1. An airbag type electric toe correction device, comprising the main support (1) with hollow interior, the main support (1) and auxiliary support (2) being detachably connected; below the joint of main support (1) and auxiliary support (2) are equipped with an airbag (3), which is connected with the pneumatic pump (4) inside the main support (1); the airbag (3) is a multi-layer structure, when the airbag (3) is inflated, adjacent two layers of the airbag (3) form V-shape gaps (20);

2. The airbag type electric toe correction device of claim 1 wherein said auxiliary support (2) comprises a fixing portion (5) and a correction portion (6), one end of said fixing portion (5) is provided with a circular opening (7) to be clamped with the column-shaped body (8) provided on the main support (1).

3. The airbag type electric toe correction device of claim 2 wherein the left and right sides of the opening (7) in said auxiliary support (2) are hollowed out to form hollow slots (9), and said auxiliary support (2) is also provided with a through strap fixing groove (10) in long strip shape, and said strap fixing groove (10) is tied with a big toe strap (21).

4. The airbag type electric toe correction device of claim 2 wherein the included angle formed by said fixing portion (5) and correction portion (6) is from 0 to 15 degrees.

5. The airbag type electric toe correction device of claim 2 wherein the included angle formed by said fixing portion (5) and correction portion (6) is from 16 to 30 degrees.

6. The airbag type electric toe correction device of claim 1 wherein said auxiliary support (2) is provided with a circular display (11); on the left and right sides of said main support (1) are symmetrically provided with strap fixing rings (16), on which are tied with foot strap (22).

7. The airbag type electric toe correction device of claim 1 wherein the air outlet of said pneumatic pump (4) is connected to the airbag (3) via air tube; said pneumatic pump (4) is connected to the circuit board (12); below the circuit board (12) is connected with the battery (13), and a control button (14) is provided above said circuit board (12).

8. The airbag type electric toe correction device of claim 7 wherein a charging port (15) is provided at the rear of said circuit board (12), said circuit board (12) fits into the remote control (19), and a solenoid valve (18) is provided on one side of said pneumatic pump (4).

9. The airbag type electric toe correction device of claim 1 wherein one side of said main support (1) is sunken inwardly so as to fit the side of the foot of human body, one side of said auxiliary support (2) is sunken inwardly so as to fit the big toe, and one side of said main support (1) is connected to a flexible and soft spacer (17), and inside said spacer (17) is provided with a vibration motor.

10. The airbag type electric toe correction device of claim 9 wherein the side of said spacer (17) that fits the foot of the human body is provided with a vibration motor, which is a micro vibration motor and located inside the spacer (17) so that the spacer (17) protrudes in the direction of foot of the human body so as to better contact the foot of the human body.
